(19) United States
(12) Patent Application Publication
(10) Pub. No.: US 2026/0074033 A1
(43) Pub. Date: Mar. 12, 2026

(54) APPARATUS AND METHOD FOR CONTROLLING PHARMACEUTICAL MIXER BASED ON SIMILAR CLINICAL TRIAL DATA EXTRACTED BY MACHINE LEARNINGS

(71) Applicant: MEDIAIPLUS, INC., Seongnam-si (KR)

(72) Inventors: Ji Hee JUNG, Seongnam-si (KR); Nam Goo SONG, Suwon-si Gyeonggi-do (KR); Yong Jang JO, Seongnam-si Gyeonggi-do (KR)

(21) Appl. No.: 19/392,282

(22) Filed: Nov. 18, 2025

Related U.S. Application Data
(63) Continuation-in-part of application No. 18/039,404, filed on May 30, 2023.

(30) Foreign Application Priority Data
Nov. 30, 2020 (KR) 10-2020-0164313

(51) Int. Cl. G16H 10/20

(52) U.S. Cl. CPC G16H 10/20

(57) ABSTRACT

An apparatus and a method for controlling a pharmaceutical mixer based on similar clinical trial data extracted by machine learnings. The method may include: training a learning model; when clinical trial data is received from a user terminal, determining a type of the clinical trial data; generating a vector using each piece of metadata of the clinical trial data; generating a vector by tokenizing words extracted from the clinical trial data according to the type of the clinical trial data; inputting the vector to the pretrained learning model and calculating a distance between a prestored vector in the learning model and the vector; measuring a similarity grade; extracting clinical trial data having a predetermined similarity grade; and transmitting a control signal to the pharmaceutical mixer based on the extracted clinical trial data.

### CROSS REFERENCE TO RELATED APPLICATION

This application a continuation-in-part application claiming priority to US non-provisional application No. Ser. No. 18/039,404 filed on May 30, 2023 claiming priority from International Patent Application No. PCT/KR2021/009978 filed on Jul. 30, 2021, which claims priority from Korea Patent Application No. 10-2020-0164313 filed on Nov. 30, 2020, which is hereby incorporated by reference in its entirety.

### TECHNICAL FIELD

The present disclosure relates to providing similar clinical trial data, and more specifically, to a similar clinical trial data provision method of extracting and providing clinical trial data which is similar to clinical trial data input by a user and a server for performing the same.

### BACKGROUND

As the biotechnology industry expands, clinical trials for developing new medicines are increasing. In general, a clinical trial may be defined as a test or study conducted on human subjects to evaluate the efficacy of a newly developed medicine or establish safety standards, check the range of applicable diseases, appropriate dosage, the range of side effects, pharmacokinetics, pharmacology, clinical effects, etc. of the corresponding medicines, etc. and examine adverse reactions or harmful drug reactions.

Such clinical trials are used through conventional case report forms (CRFs). Clinical trials are being used to objectively and experientially verify the hypothesis or purpose of a clinical trial by recording several interviews, drug administration, examination, and evaluation of a large number of subjects and data collected from the process on paper media and statistically analyzing the data.

However, such paper media-based clinical trial data management not only involves extreme difficulty in data storage, maintenance, and security but also has inherent problems such as extremely limited data sharing, data reprocessing, variability or fluidity of test or review period, follow-up reference, utilization, etc.

Recently, to solve this problem, some electronic data-based clinical trial management systems (electronic CRF (eCRF) systems) have been disclosed. Such a clinical trial management system includes a clinical data database for storing clinical trial data.

Meanwhile, a clinical trial management system provides clinical data stored in a clinical data database to clinical researchers. Accordingly, researchers conducting clinical research search for necessary items in consideration of their research subjects.

### SUMMARY

### Technical Problem

The present disclosure is directed to providing a similar clinical trial data provision method of extracting and providing clinical trial data which is similar to clinical trial data input by a user and a server for performing the same.

Technical problems to be solved by disclosure are not limited to that described above. Other technical problems and advantages of the present disclosure which have not been described will be understood from the following description and more clearly understood through embodiments of the present disclosure. Also, it will be readily seen that the technical problems and advantages of the present disclosure may be achieved by means described in the claims and combinations thereof.

### Technical Solution

One aspect of the present disclosure provides a method of providing similar clinical trial data performed by a similar clinical trial data provision server, the method including, when clinical trial data is received from a user terminal, determining a type of the clinical trial data, generating a vector using each piece of metadata of the clinical trial data or generating a vector by tokenizing words extracted from the clinical trial data according to the type of the clinical trial data, inputting the vector to a pretrained learning model and calculating a distance between a prestored vector in the learning model and the vector, and measuring a similarity grade according to the distance between the vectors and extracting and providing clinical trial data having a similarity grade which is lower than or equal to a specific grade.

Another aspect of the present disclosure provides a similar clinical trial data provision device including a preprocessing unit configured to determine, when clinical trial data is received from a user terminal, a type of the clinical trial data and preprocess the clinical trial data according to the type of the clinical trial data, a data feature extraction unit configured to generate a vector using each piece of metadata of the clinical trial data or generate a vector by tokenizing words extracted from the clinical trial data, and a similar clinical trial data extraction unit configured to input the vector to a pretrained learning model, calculate a distance between a prestored vector in the learning model and the vector, measure a similarity grade according to the distance between the vectors, and extract and provide clinical trial data having a similarity grade which is lower than or equal to a specific grade.

Further, another aspect of the present disclosure provides a computer-implemented method for controlling a pharmaceutical mixer based on similar clinical trial data extracted by machine learnings. The method may include: collecting a set of clinical trial data from a database; determining a type of each clinical trial data of the set of clinical trial data; preprocessing the set of clinical trial data according to the type of each clinical trial data; generating a first vector set using metadata of the set of clinical trial data according to the type of each clinical trial data; training a learning model in a first stage using the first vector set; generating a second vector set by tokenizing words extracted from the set of clinical trial data; training the learning model in a second stage using the second vector set; when clinical trial data is received from a user terminal, determining a type of the clinical trial data; generating a vector using each piece of metadata of the clinical trial data; generating a vector by tokenizing words extracted from the clinical trial data according to the type of the clinical trial data; inputting the vector to the pretrained learning model and calculating a distance between a prestored vector in the learning model and the vector; measuring a similarity grade according to the distance between the vectors; extracting clinical trial data having a similarity grade which is lower than or equal to a predetermined grade; and transmitting a control signal to the pharmaceutical mixer based on the extracted clinical trial data.

According to an exemplary embodiment, the generating of the vector by tokenizing the words extracted from the clinical trial data according to the type of the clinical trial data comprises: when the type of the clinical trial data is unstructured data, deleting predetermined clinical non-use words from clinical title data and extracting words from the clinical title data from which the predetermined clinical non-use words are deleted on the basis of a blank; performing morpheme analysis on each of the words and generating tokens each of which includes a pair of a word and a morpheme value and is assigned a label indicating a frequency; and generating a documentary word matrix by giving a different weight to each of the tokens according to words and labels of the tokens. Also, in an exemplary embodiment, the generating of the documentary word matrix by giving the different weight to each of the tokens according to the words and labels of the tokens comprises: decomposing the documentary word matrix into a first matrix having a size of (the number of pieces of clinical trial data×k which is the number of topics) and a second matrix having a size of (k which is the number of topics×the number of words) through a non-negative matrix factorization machine learning algorithm; and updating the first matrix and second matrix by clustering the clinical trial data and each of the words into any one of the k topics.

Also, according to an exemplary embodiment, the generating of the vector using each piece of metadata of the clinical trial data and the generating of the vector by tokenizing the words extracted from the clinical trial data according to the type of the clinical trial data comprises generating a sub-vector for each piece of metadata of the clinical trial data and generating a vector using sub-vectors for the metadata when the type of the clinical trial data is structured data.

### Advantageous Effects

According to the above-described present disclosure, it is possible to extract and provide clinical trial data which is similar to clinical trial data input by a user.

### BRIEF DESCRIPTION OF DRAWINGS

FIG. 1 is a network configuration diagram illustrating a system for providing similar clinical trial data according to an embodiment of the present disclosure.

FIG. 2 is a block diagram illustrating an internal structure of a server for providing similar clinical trial data according to an embodiment of the present disclosure.

FIG. 3 is a flowchart illustrating a method of providing similar clinical trial data according the present disclosure.

FIG. 4 is a flowchart illustrating a method of providing similar clinical trial data according to another embodiment of the present disclosure.

FIG. 5 is a flowchart illustrating a method for controlling a pharmaceutical mixer based on similar clinical trial data extracted by machine learnings according to another embodiment of the present disclosure.

FIG. 6 is a block diagram illustrating that a control signal is transmitted to a pharmaceutical mixer based on based on similar clinical trial data extracted by machine learnings according to another embodiment of the present disclosure.

### DETAILED DESCRIPTIONS OF EXEMPLARY EMBODIMENTS

The foregoing technical problems, features, and advantages will be described in detail below with reference to the accompanying drawings. Accordingly, those skilled in the technical field to which the present disclosure pertains may readily implement the technical spirit of the present disclosure. In describing the present disclosure, when the detailed description of a well-known technology related to the present disclosure is determined to unnecessarily obscure the subject matter of the present disclosure, the detailed description will be omitted. Hereinafter, exemplary embodiments of the present disclosure will be described in detail with reference to the accompanying drawings. Throughout the drawings, like reference numerals refer to like components.

Among terms used herein, the term “clinical trial data” means data collected through a web or database and includes unstructured data and structured data.

Structured data is data including metadata such as a current research information system (CRIS) registration number, a Korean abstract title, an English abstract title, an approval state, an approval date, etc., and unstructured data is a data list in natural language such as clinical trial results.

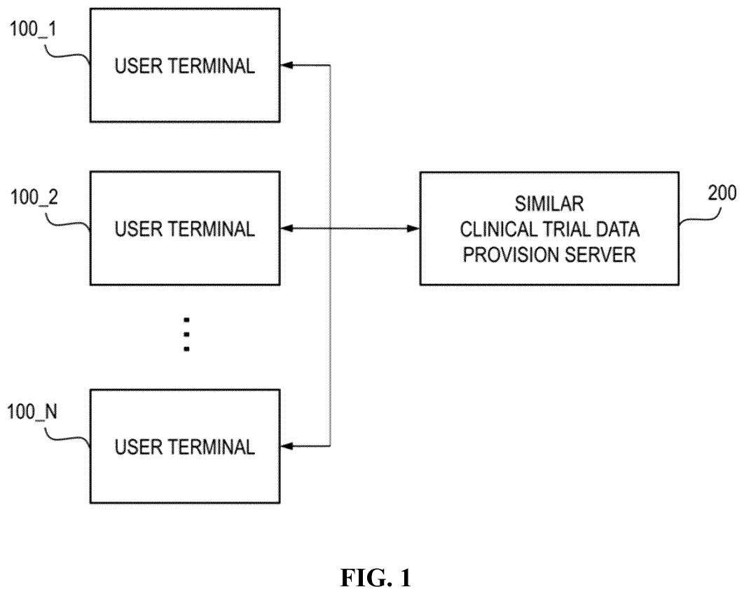

FIG. 1 is a network configuration diagram illustrating a system for providing similar clinical trial data according to an embodiment of the present disclosure.

Referring to FIG. 1, the system for providing similar clinical trial data according to an embodiment of the present disclosure includes user terminals 100_1 to 100_N and a similar clinical trial data provision server 200.

The user terminals 100_1 to 100_N are terminals held by users to provide clinical trial data to the similar clinical trial data provision server 200 and receive clinical trial data similar to the clinical trial data from the similar clinical trial data provision server 200. Each of the user terminals 100_1 to 100_N may be implemented as a smartphone, a tablet personal computer (PC), a laptop computer, a desktop computer, etc.

The similar clinical trial data provision server 200 is a server that receives clinical trial data from the user terminals 100_1 to 100_N and extracts and provides clinical trial data similar to the received clinical trial data.

To this end, the similar clinical trial data provision server 200 collects clinical trial data through a web or a clinical trial database and preprocesses the clinical trial. Here, the similar clinical trial data provision server 200 performs different types of preprocessing depending on whether the clinical trial data is structured data or unstructured data.

According to an embodiment, when the clinical trial data is structured data, the similar clinical trial data provision server 200 generates a sub-vector for each piece of metadata of the clinical trial data and generates a vector using sub-vectors for the metadata.

The similar clinical trial data provision server 200 normalizes or preprocesses a weight calculated through the above-described process into another form, such as term frequency-inverse document frequency (TF-IDF), and then generates a learning model through training with the vector. When structured clinical trial data is received later from the user terminals 100_1 to 100_N, the learning model allows extraction of clinical trial data similar to the received clinical trial data.

According to another embodiment, when the clinical trial data is unstructured data, the similar clinical trial data provision server 200 may delete predetermined clinical non-use words from the clinical trial data or delete predetermined clinical non-use parts of speech. Here, the predetermined clinical non-use parts of speech may include articles, prepositions, conjunctions, exclamations, etc.

For example, when clinical trial data “A Randomized, Double Blind Trial of LdT (Telbivudine) Versus Lamivudine in Adults With Compensated Chronic Hepatitis B” is received, the similar clinical trial data provision server 200 deletes “A,” “of,” “in,” “with,” and “B” which are predetermined clinical non-use words.

After that, the similar clinical trial data provision server 200 extracts words from the clinical trial data from which the predetermined clinical non-use words are deleted on the basis of blanks and measures frequencies of the words in the clinical trial data.

Subsequently, the similar clinical trial data provision server 200 performs morpheme analysis of each word to generate a token which includes a pair of a word and a morpheme value and is assigned a label indicating a frequency.

For example, the similar clinical trial data provision server 200 may generate tokens, such as (frequency: 1000, (a word, a morpheme value)), (frequency: 234, (a word, a morpheme)), (frequency: 2541, (a word, a morpheme)), (frequency: 2516, (a word, a morpheme)), etc., from the clinical trial data from which the predetermined clinical non-use words are deleted.

After the tokens are generated as described above on the basis of the clinical trial data from which the predetermined clinical non-use words are deleted, the similar clinical trial data provision server 200 assigns a different weight to each of the tokens according to words and labels of the tokens.

According to an embodiment, the similar clinical trial data provision server 200 assigns a different weight to each of the tokens according to types of languages (i.e., English, Chinese, Korean, etc.) corresponding to words of the tokens, positions of the words in the clinical trial data, and frequencies of the labels assigned to the tokens, thereby generating a documentary word matrix.

Subsequently, the similar clinical trial data provision server 200 decomposes the documentary word matrix into a matrix having a size of (the number of pieces of clinical trial data×k) and a matrix having a size of (k×the number of words) through a non-negative matrix factorization machine learning algorithm. Here, the integer k is a hyperparameter (i.e., a topic number) and may be determined to be the number of topics to be clustered. For example, k may be determined to be the number of diseases or the like.

Through the above process, the clinical trial data and each of the words may be clustered into any one of the k topics so that the first matrix and the second matrix may be updated.

Subsequently, the similar clinical trial data provision server 200 generates a learning model using the first matrix and the second matrix. When unstructured clinical trial data is received later from the user terminals 100_1 to 100_N, the learning model may allow extraction of clinical trial data similar to the received clinical trial data.

A process of extracting clinical trial data similar to clinical trial data using a learning model will be described below.

First, when clinical trial data is received from the user terminals 100_1 to 100_N, the similar clinical trial data provision server 200 vectorizes the clinical trial data through the above-described process according to the type of clinical trial data.

Subsequently, the similar clinical trial data provision server 200 may calculate a distance between a matrix generated on the basis of the clinical trial data received from the user terminals 100_1 to 100_N and a matrix of the learning model, thereby calculating a similarity between clinical trial data.

After the above process, the similar clinical trial data provision server 200 may extract and provide similar clinical trial data according to a distance between a vector of the learning model and a vector generated on the basis of the clinical trial data received from the user terminals 100_1 to 100_N.

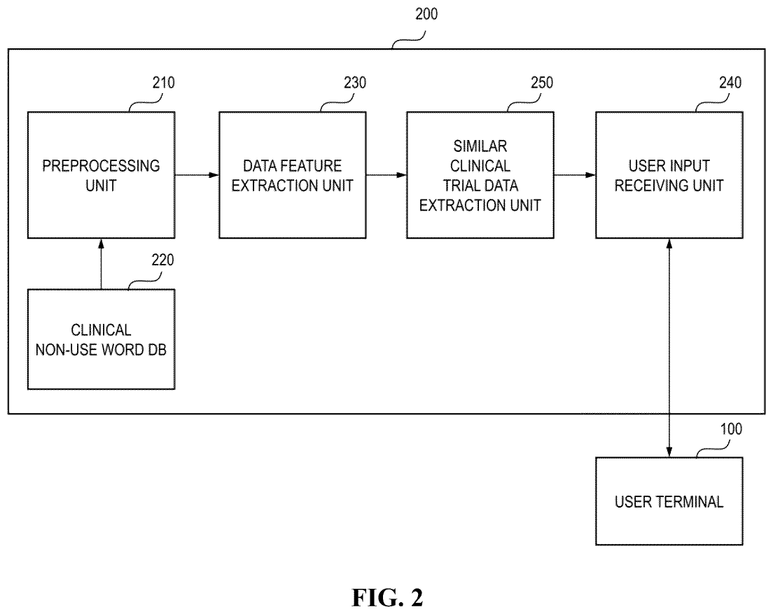

FIG. 2 is a block diagram illustrating an internal structure of a server for providing similar clinical trial data according to an embodiment of the present disclosure.

Referring to FIG. 2, the similar clinical trial data provision server 200 includes a preprocessing unit 210, a clinical non-use word database 220, a data feature extraction unit 230, a user input receiving unit 240, and a similar clinical trial data extraction unit 250.

The preprocessing unit 210 collects clinical trial data through a web or a clinical trial database and preprocesses the clinical trial data. Here, the preprocessing unit 210 performs different types of preprocessing depending on whether the clinical trial data is structured data or unstructured data.

According to an embodiment, when the clinical trial data is structured data, the preprocessing unit 210 extracts metadata of the clinical trial data.

Subsequently, the preprocessing unit 210 generates a learning model through training with a vector. When structured clinical trial data is received later from the user terminals 100_1 to 100_N, the learning model allows extraction of clinical trial data similar to the received clinical trial data.

According to another embodiment, when the clinical trial data is unstructured data, the preprocessing unit 210 deletes predetermined clinical non-use words from the clinical trial data or deletes predetermined clinical non-use parts of speech. Here, the predetermined clinical non-use parts of speech may include articles, prepositions, conjunctions, exclamations, etc.

For example, when clinical trial data “A Randomized, Double Blind Trial of LdT (Telbivudine) Versus Lamivudine in Adults With Compensated Chronic Hepatitis B” is received, the preprocessing unit 210 deletes “A,” “of,” “in,” “with,” and “B” which are predetermined clinical non-use words.

After that, the preprocessing unit 210 extracts words from the clinical trial data from which the predetermined clinical non-use words are deleted on the basis of blanks and measures frequencies of the words in the clinical trial data.

Subsequently, the preprocessing unit 210 performs morpheme analysis of each word to generate a token which includes a pair of a word and a morpheme value and is assigned a label indicating a frequency.

For example, the preprocessing unit 210 may generate tokens, such as (frequency: 1000, (a word, a morpheme value)), (frequency: 234, (a word, a morpheme)), (frequency: 2541, (a word, a morpheme)), (frequency: 2516, (a word, a morpheme)), etc., from the clinical trial data from which the predetermined clinical non-use words are deleted.

The data feature extraction unit 230 generates a learning model using information generated by the preprocessing unit 210.

According to an embodiment, the data feature extraction unit 230 generates a sub-vector using each piece of the metadata extracted by the preprocessing unit 210 and generates a vector using the sub-vectors for the metadata.

According to another embodiment, the data feature extraction unit 230 assigns a different weight to each of the tokens generated by the preprocessing unit 210 according to words and labels of the tokens.

In other words, the data feature extraction unit 230 assigns a different weight to each of the tokens according to types of languages (i.e., English, Chinese, Korean, etc.) corresponding to words of the tokens, positions of the words in the clinical trial data, and frequencies of the labels assigned to the tokens, thereby generating a documentary word matrix.

First, the data feature extraction unit 230 calculates a first weight using the total number of tokens generated from a clinical trial title and the order of the tokens on the basis of [Equation 1] below.

$$w 1 = \frac{{token}_{i}}{token ( {input}_{data} )} \times L \qquad [ Equation 1 ]$$

$$W 1 : a first weight of a token ,$$

$$input\_data : a clinical trial title ,$$

$$token ( ) a function for returning the total number of tokens after a$$

$$clinical trial title is tokenized ,$$

$$token\_i : the number of the token among the total number of tokens ,$$

$$i : a number indicating the position of a token , and$$

$$L : an important value predetermined according to the type of language$$

$$In other words , the data feature extraction unit 230 calculates a first$$

$$weight according to the position of a token among the total number of$$

$$tokens and an important value predetermined according to the type of$$

$$language on the basis of [ Equation 1 ] .$$

For example, when the total number of tokens is 12 and the order of a token is fourth, the data feature extraction unit 230 may calculate “0.25” and then calculate a first weight by applying an important value predetermined according to the type of language to the calculated value.

Here, the important value predetermined according to the type of language may change depending on a position at which an important word is present according to the type of language. In other words, the important value predetermined according to the type of language may change depending on the number of a current token.

After that, the data feature extraction unit 230 may calculate a second weight for each token using a frequency indicated by a label preassigned to the token and frequencies indicated by labels preassigned to the preceding token and the subsequent token on the basis of [Equation 2] and [Equation 3] below.

$${Difference}_{value} = \frac{f ( {token}_{i} - 1 ) + f ( {token}_{i} ) + f ( {token}_{i} + 1 )}{3} \qquad [ Equation 2 ]$$

$$Difference\_value : the average of frequencies$$

$$token\_i : an {i}^{th} token among the total number of tokens ,$$

$$token\_i - 1 : the token preceding the {i}^{th} token among the total number$$

$$of tokens ,$$

$$token\_i + 1 : the token subsequent to the {i}^{th} token among the total$$

$$number of tokens ,$$

$$f ( ) a function for extracting a frequency indicated by a label assigned$$

$$to a token , and$$

$$i : a number indicating a position of a token$$

$$If ( Difference\_Value > Threshold ) , \\ W 2 = 0 Else ( Difference\_Value < Threshold ) , W 2 = 1 \qquad [ Equation 3 ]$$

$$W 2 : a second weight of a token ,$$

$$Difference\_Value : the average of frequencies$$

$$calculated with [ Equation 2 ]$$

$$Threshold : a threshold value$$

As described above, the data feature extraction unit 230 calculates a first weight and a second weight on the basis of [Equation 1] to [Equation 3], calculates a final weight using the first weight and the second weight, and then assigns the final weight, thereby generating a documentary word matrix.

After that, the data feature extraction unit 230 decomposes the documentary word matrix into a matrix having a size of (the number of pieces of clinical trial data×k) and a matrix having a size of (k×the number of words) through a non-negative matrix factorization machine learning algorithm. Here, the integer k is a hyperparameter (i.e., a topic number) and may be determined to be the number of topics to be clustered. For example, k may be determined to be the number of diseases or the like.

Through the above process, the clinical trial data and each of the words may be clustered into any one of the k topics so that the first matrix and the second matrix may be updated.

Subsequently, the data feature extraction unit 230 generates a learning model using the first matrix and the second matrix. When unstructured clinical trial data is received later from the user terminals 100_1 to 100_N, the learning model may allow extraction of clinical trial data similar to the received clinical trial data.

When the user input receiving unit 240 receives clinical trial data from the user terminals 100_1 to 100_N, the preprocessing unit 210 and the data feature extraction unit 230 perform preprocessing and data feature extraction according to the type of clinical trial data.

When a vector is extracted from the clinical trial data received from the user terminals 100_1 to 100_N through the preprocessing unit 210 and the data feature extraction unit 230, the similar clinical trial data extraction unit 250 inputs the vector to the pretrained learning model.

Through the learning model, the similar clinical trial data extraction unit 250 calculates a distance between a prestored vector in the learning model and the vector, measures a similarity grade according to the distance between the vectors, and extracts and provides clinical trial data having a similarity grade which is lower than or equal to a specific grade.

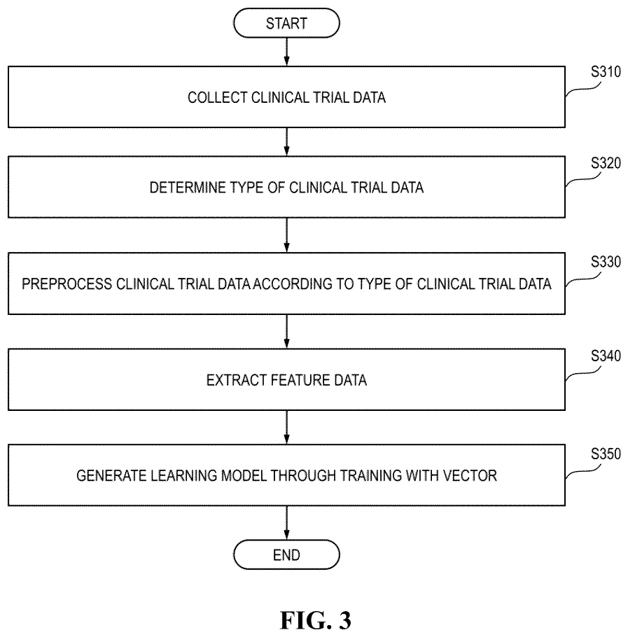

FIG. 3 is a flowchart illustrating a method of providing similar clinical trial data according the present disclosure.

Referring to FIG. 3, the similar clinical trial data provision server 200 collects clinical trial data through a web or a clinical trial database (operation S310), determines the type of clinical trial data (operation S320), and preprocesses the clinical trial data according to the type of clinical trial data (operation S330).

The similar clinical trial data provision server 200 generates a vector using each piece of metadata of the clinical trial data according to the type of clinical trial data or generates a vector by tokenizing words extracted from the clinical trial data (operation S340).

The similar clinical trial data provision server 200 generates a learning model through training with the vector (operation S350).

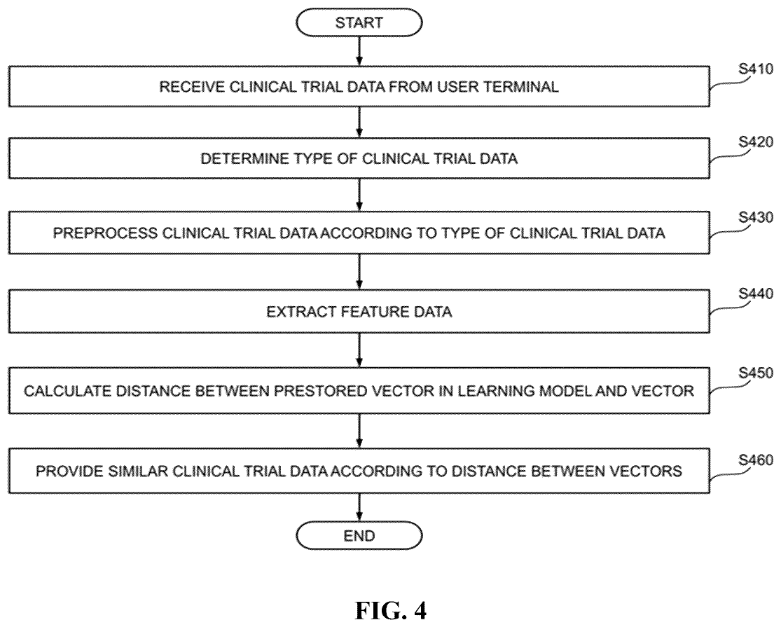

FIG. 4 is a flowchart illustrating a method of providing similar clinical trial data according to another embodiment of the present disclosure.

Referring to FIG. 4, when clinical trial data is received from a user terminal (operation S410), the similar clinical trial data provision server 200 determines the type of clinical trial data (operation S420) and preprocesses the clinical trial data according to the type of clinical trial data (operation S430).

The similar clinical trial data provision server 200 generates a vector using each piece of metadata of the clinical trial data according to the type of clinical trial data or generates a vector by tokenizing words extracted from the clinical trial data (operation S440).

The similar clinical trial data provision server 200 inputs the vector to a pretrained learning model and calculates a distance between a prestored vector in the learning model and the vector (operation S450).

The similar clinical trial data provision server 200 measures a similarity grade according to the distance between the vectors and extracts and provides clinical trial data having a similarity grade which is lower than or equal to a specific grade (operation S460).

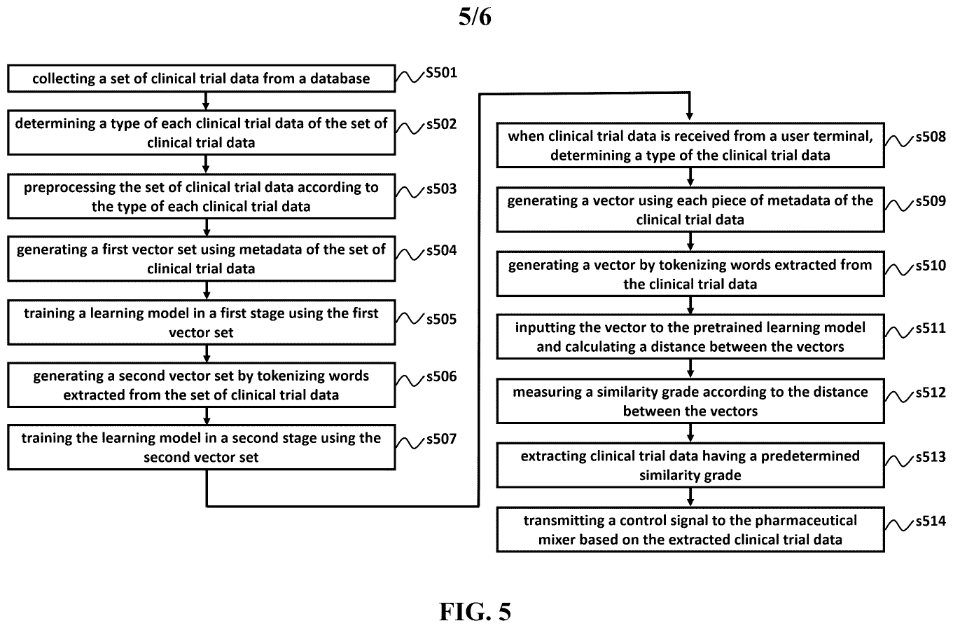

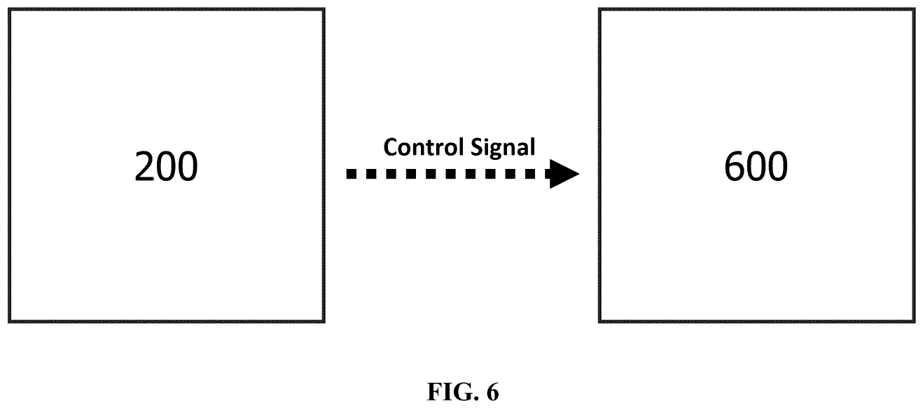

Turning now to FIGS. 5 and 6, FIG. 5 is a flowchart illustrating a method for controlling a pharmaceutical mixer based on similar clinical trial data extracted by machine learnings, and FIG. 6 is a block diagram illustrating that a control signal is transmitted to a pharmaceutical mixer based on based on similar clinical trial data extracted by machine learnings. According to an exemplary embodiment, a computer-implemented method may be provided for controlling a pharmaceutical mixer based on similar clinical trial data extracted by machine learnings. The method may include: collecting a set of clinical trial data from a database (operation S501); determining a type of each clinical trial data of the set of clinical trial data (operation S502); preprocessing the set of clinical trial data according to the type of each clinical trial data (operation S503); generating a first vector set using metadata of the set of clinical trial data according to the type of each clinical trial data (operation S504); training a learning model in a first stage using the first vector set (operation S505); generating a second vector set by tokenizing words extracted from the set of clinical trial data (operation S506); training the learning model in a second stage using the second vector set (operation S507); when clinical trial data is received from a user terminal, determining a type of the clinical trial data (operation S508); generating a vector using each piece of metadata of the clinical trial data (operation S509); generating a vector by tokenizing words extracted from the clinical trial data according to the type of the clinical trial data (operation S510); inputting the vector to the pretrained learning model and calculating a distance between a prestored vector in the learning model and the vector (operation S511); measuring a similarity grade according to the distance between the vectors (operation S512); extracting clinical trial data having a similarity grade which is lower than or equal to a predetermined grade (operation S513); and transmitting a control signal to the pharmaceutical mixer 600 based on the extracted clinical trial data (operation S514).

Also, as similar as the above discussed exemplary embodiments, the generating of the vector by tokenizing the words extracted from the clinical trial data according to the type of the clinical trial data comprises: when the type of the clinical trial data is unstructured data, deleting predetermined clinical non-use words from clinical title data and extracting words from the clinical title data from which the predetermined clinical non-use words are deleted on the basis of a blank; performing morpheme analysis on each of the words and generating tokens each of which includes a pair of a word and a morpheme value and is assigned a label indicating a frequency; and generating a documentary word matrix by giving a different weight to each of the tokens according to words and labels of the tokens. Also, in an exemplary embodiment, the generating of the documentary word matrix by giving the different weight to each of the tokens according to the words and labels of the tokens comprises: decomposing the documentary word matrix into a first matrix having a size of (the number of pieces of clinical trial data×k which is the number of topics) and a second matrix having a size of (k which is the number of topics×the number of words) through a non-negative matrix factorization machine learning algorithm; and updating the first matrix and second matrix by clustering the clinical trial data and each of the words into any one of the k topics. Further, according to an exemplary embodiment, the generating of the vector using each piece of metadata of the clinical trial data and the generating of the vector by tokenizing the words extracted from the clinical trial data according to the type of the clinical trial data comprises generating a sub-vector for each piece of metadata of the clinical trial data and generating a vector using sub-vectors for the metadata when the type of the clinical trial data is structured data.

The embodiments described above can be implemented in a form of an executable program command through a variety of computer means recordable to computer readable media. The computer readable media may include solely or in combination, program commands, data files, and data structures.

The program commands recorded to the media may be components specially designed for the present invention or may be usable by a skilled person in a field of computer software.

Computer readable recording media include magnetic media such as a hard disk, a floppy disk, magnetic tape, an optical media such as a CD-ROM and a DVD, a magneto-optical media such as a floptical disk and hardware devices such as ROM, RAM, and flash memory specially designed to store and carry out programs. Program commands include not only a machine language code made by a complier, but also a high level code that can be used by an interpreter etc., which is executed by a computer. The aforementioned hardware device can work as more than a software module to perform the action of the present invention, and they can do the same in the opposite case.

Aspects of the present disclosure may take a form of hardware overall, software (including firmware, resident software, micro codes, or the like) overall, or computer program products embodied in at least one computer readable medium on which computer readable program codes are implemented.

Although the present disclosure has been described with reference to limited embodiments and drawings, the present disclosure is not limited to the embodiments. Various alterations and modifications can be made by those of ordinary skill in the art to which the present disclosure pertains. Therefore, the spirit of the present disclosure should be determined by only the following claims, and all equivalents or equivalent modifications thereof fall within the scope of the present disclosure.

1. A computer-implemented method for controlling a pharmaceutical mixer based on similar clinical trial data extracted by machine learnings, the method comprising:
collecting a set of clinical trial data from a database;
determining a type of each clinical trial data of the set of clinical trial data;
preprocessing the set of clinical trial data according to the type of each clinical trial data;
generating a first vector set using metadata of the set of clinical trial data according to the type of each clinical trial data;
training a learning model in a first stage using the first vector set;
generating a second vector set by tokenizing words extracted from the set of clinical trial data;
training the learning model in a second stage using the second vector set;
when clinical trial data is received from a user terminal, determining a type of the clinical trial data;
generating a vector using each piece of metadata of the clinical trial data;
generating a vector by tokenizing words extracted from the clinical trial data according to the type of the clinical trial data;
inputting the vector to the pretrained learning model and calculating a distance between a prestored vector in the learning model and the vector;
measuring a similarity grade according to the distance between the vectors;
extracting clinical trial data having a similarity grade which is lower than or equal to a predetermined grade; and
transmitting a control signal to the pharmaceutical mixer based on the extracted clinical trial data,
wherein the generating of the vector by tokenizing the words extracted from the clinical trial data according to the type of the clinical trial data comprises:
when the type of the clinical trial data is unstructured data, deleting predetermined clinical non-use words from clinical title data and extracting words from the clinical title data from which the predetermined clinical non-use words are deleted on the basis of a blank;
performing morpheme analysis on each of the words and generating tokens each of which includes a pair of a word and a morpheme value and is assigned a label indicating a frequency; and
generating a documentary word matrix by giving a different weight to each of the tokens according to words and labels of the tokens,
wherein the generating of the documentary word matrix by giving the different weight to each of the tokens according to the words and labels of the tokens comprises:
decomposing the documentary word matrix into a first matrix having a size of (the number of pieces of clinical trial data×k which is the number of topics) and a second matrix having a size of (k which is the number of topics×the number of words) through a non-negative matrix factorization machine learning algorithm; and
updating the first matrix and second matrix by clustering the clinical trial data and each of the words into any one of the k topics.

2. The method of claim 1, wherein the generating of the vector using each piece of metadata of the clinical trial data and the generating of the vector by tokenizing the words extracted from the clinical trial data according to the type of the clinical trial data comprises:
when the type of the clinical trial data is structured data, generating a sub-vector for each piece of metadata of the clinical trial data and generating a vector using sub-vectors for the metadata.

3. An apparatus for controlling a pharmaceutical mixer based on similar clinical trial data extracted by machine learnings, the apparatus comprising a processor and one or more memory devices communicatively coupled to the processor, and the one or more memory devices stores instructions operable when executed by the processor to perform the steps of:
collecting a set of clinical trial data from a database;
determining a type of each clinical trial data of the set of clinical trial data;
preprocessing the set of clinical trial data according to the type of each clinical trial data;
generating a first vector set using metadata of the set of clinical trial data according to the type of each clinical trial data;
training a learning model in a first stage using the first vector set;
generating a second vector set by tokenizing words extracted from the set of clinical trial data;
training the learning model in a second stage using the second vector set;
when clinical trial data is received from a user terminal, determining a type of the clinical trial data;
generating a vector using each piece of metadata of the clinical trial data;
generating a vector by tokenizing words extracted from the clinical trial data according to the type of the clinical trial data;
inputting the vector to the pretrained learning model and calculating a distance between a prestored vector in the learning model and the vector;
measuring a similarity grade according to the distance between the vectors;
extracting clinical trial data having a similarity grade which is lower than or equal to a predetermined grade; and
transmitting a control signal to the pharmaceutical mixer based on the extracted clinical trial data,
wherein, when the type of the clinical trial data is unstructured data, predetermined clinical non-use words are deleted from clinical title data, extracts words from the clinical title data from which the predetermined clinical non-use words are deleted on the basis of a blank, generates tokens each of which includes a pair of a word and a morpheme value and is assigned a label indicating a frequency by performing morpheme analysis on each of the words, and generates a documentary word matrix by giving a different weight to each of the tokens according to words and labels of the tokens, and
wherein a documentary word matrix is decomposed into a first matrix having a size of (the number of pieces of clinical trial data×k which is the number of topics) and a second matrix having a size of (k which is the number of topics×the number of words) through a non-negative matrix factorization machine learning algorithm and updates the first matrix and second matrix by clustering the clinical trial data and each of the words into any one of the k topics.

4. The apparatus of claim 3, wherein, when the type of the clinical trial data is structured data, a sub-vector is generated for each piece of metadata of the clinical trial data, and a vector is generated using sub-vectors for the metadata.
